(19) United States
(12) Patent Application Publication
(10) Pub. No.: US 2011/0190774 A1
(43) Pub. Date: Aug. 4, 2011

(54) METHODS AND APPARATUS FOR PERFORMING AN ARTHROSCOPIC PROCEDURE USING SURGICAL NAVIGATION

(76) Inventors: Julian Nikolchev, Portola Valley, CA (US); James R. Flom, San Carlos, CA (US)

(21) Appl. No.: 12/949,422

(22) Filed: Nov. 18, 2010

Related U.S. Application Data
(60) Provisional application No. 61/262,196, filed on Nov. 18, 2009.

(51) Int. Cl. A61B 17/56

(52) U.S. Cl. 606/90; 606/86 R

(57) ABSTRACT

The method for performing an arthroscopic procedure on a joint using surgical navigation. A 3-D virtual model of the anatomy is created from a scan of the anatomy. The 3-D virtual model is then used to reproduce motion of the joint and plan the arthroscopic procedure. The 3-D virtual model is placed into registration with the real-world anatomy, so that a virtual image generated by the 3-D virtual model may be placed into registration with an arthroscopic image of the real-world anatomy. Preferably, arthroscopic registration markers, positioned prior to scanning, are used to place the 3-D virtual model in registration with the real-world anatomy. The arthroscopic registration markers may be placed either percutaneously or arthroscopically. At the conclusion of the procedure, the registration markers may be left in place, removed arthroscopically or allowed to biodegrade.

### REFERENCE TO PENDING PRIOR PATENT APPLICATIONS

This patent application claims benefit of pending prior U.S. Provisional Patent Application Ser. No. 61/262,196, filed Nov. 18, 2009 by Julian Nikoichev et al. for METHOD AND APPARATUS FOR SURGICAL NAVIGATION FOR HIP ARTHROSCOPY (Attorney's Docket No. FIAN-50 PROV), which patent application is hereby incorporated herein by reference.

### FIELD OF THE INVENTION

This invention relates to surgical methods and apparatus in general, and more particularly to surgical methods and apparatus for arthroscopically treating the hip.

### BACKGROUND OF THE INVENTION

One of many problems that can affect the function of the hip joint is a decreased range of motion. One common pathology is femoral acetabular impingement (FAT). FAI may occur in two forms. The first form of FAI is called CAM impingement. CAM impingement exists where the head and/or neck of the femur is abnormally shaped, but the acetabular cup is normally shaped. The second form of FAI is called pincer impingement. Pincer impingement occurs where the acetabular cup is abnormally shaped, but the head and neck of the femur are normally shaped. It is also possible for an individual to simultaneously have both CAM impingement and pincer impingement. In any case, such impingement constrains the range of motion of the hip joint. See FIGS. 1A and 1B which illustrate the general anatomy of the hip joint.

Acetabular impingement can be relieved by performing a surgical procedure to remove the impinging tissue. This procedure is sometimes referred to as decompression and/or debridement.

Traditionally, acetabular impingement has been treated via open surgery, during which the surgeon removes tissue (e.g., from bone) from the femur and/or the acetabular cup so as to restore a full range of motion to the joint. However, such open surgery requires significant recovery times and causes substantial pain for the patient.

It is also possible to perform the procedure arthroscopically, however, the arthroscopic approach is extremely challenging due to the tight spaces of the hip joint, which restrict visualization of the surgical site and manipulation of surgical instruments about the surgical site. Among other things, it can be difficult for the surgeon to precisely identify the impinging bone which needs to be removed in the arthroscopic procedure. Due to these difficulties, surgeons often remove too much tissue, or too little tissue, during an arthroscopic decompression.

Surgical navigation, which involves using a three dimensional (3-D) virtual model of the anatomy, has been used with open surgery on the hip joint to help the surgeon navigate during the procedure. See, for example, U.S. Patent Publication No. 2010/0049493 (Haimerl).

Others have attempted to apply existing surgical navigation systems to minimally invasive arthroscopic hip procedures.

U.S. Patent Publication No. 2007/0249967 (Buly et al.) describes using surgical navigation to aid in treating acetabular impingement of the hip joint. The procedure described by Buly et al. involves using a probe to identify, and map, multiple points on various surfaces of the hip joint so as to create a 3-D virtual model of the anatomy. However, this procedure is tedious and time-consuming. Furthermore, it is impractical to do arthroscopically because of the limited access to the interior of the hip joint. In addition, the procedure of Buly et al. requires that a reference body be placed through the skin of the patient and into a bone prior to 3-D imaging in order to serve as a registration marker. Thereafter, the registration marker must be located in the identical position during the actual arthroscopic procedure in order to “register” the 3-D virtual model with the patient anatomy (see 100 and 110 in FIG. 1 of U.S. Patent Publication No. 2007/0249967). This means that either the reference body must protrude through the skin of the patient's leg for the period of time between when the patient's anatomy is scanned and the surgical procedure is performed, or the reference body may be removed after scanning but then must be replaced in exactly the same position prior to, or during, the arthroscopic procedure. If the reference body is not placed in exactly the same position that it occupied during scanning, the 3-D virtual model will not be properly registered with the patient anatomy and the image generated from the 3-D virtual model will not be in proper registration with the live image generated by the arthroscope during the procedure. This could result in the surgeon removing too much tissue, not enough tissue, or the wrong tissue during the arthroscopic procedure.

In “Computer-aided navigation for arthroscopic hip surgery using encoder linkages for position tracking” (Monahan et al., Int J Med Robotics Comput Assist Surq 2006; 2: 271—-278), Monahan et al. describe a surgical navigation system used in connection with an arthroscopic procedure on a hip joint. However, the procedure of Monahan et al. requires that a potentially cumbersome mechanical linkage apparatus be connected to the arthroscope and tools during the procedure. The approach of Monahan et al. also requires that a pin be placed through the skin of the patient and into an underlying bone prior to 3-D imaging in order to serve as a registration marker. Again, the registration marker used during scanning must be located in the identical position during the actual arthroscopic procedure in order to properly “register” the 3-D virtual model, with the real-world anatomy (see lines 18-20 on page 273 of the publication). Again, this means that either the pin must protrude through the skin of the patient's leg for the duration of time between scanning and the arthroscopic procedure, or the pin may be removed after scanning and then must be replaced in exactly the same position as it occupied during scanning. If the pin is not placed in exactly the same position that it occupied during scanning, the 3-D virtual model will not be in proper registration with the anatomy, which could result in the surgeon removing too much tissue, not enough tissue, or the wrong tissue during the arthroscopic procedure.

In “Evaluation of a Computed Tomography-Based Navigation System Prototype for Hip Arthroscopy in the Treatment of Femoroacetabular Cam Impingement” (Brunner et al., Arthroscopy: The Journal of Arthroscopic and Related Surgery, Vol 25, No 4 (April), 2009: pp 382-391), Brunner et al, describe a surgical navigation system used in connection with an arthroscopic procedure on the hip joint. However, this procedure also requires that a registration pin be placed through the skin of the patient and into the underlying bone (see FIG. 3 of the publication), with the pin protruding through the skin of the patient after it is emplaced. This pin must be placed into the patient prior to 3-D imaging of the anatomy, and then must either remain in place until the arthroscopic procedure is performed or the pin be replaced in exactly the same location prior to the arthroscopic procedure.

A 3-D scan of the hip may be taken several hours, days, or even weeks before an arthroscopic procedure is performed. Because of this, it is not desirable to have a reference pin protruding from the patient's leg during this time, since this can expose the patient to the risk of infection, cause trauma to the tissue if the pin should be bumped, etc.

Furthermore, in practice, it is also extremely difficult, if not impossible, to remove the reference pin after scanning and thereafter replace it, prior to the arthroscopic procedure, in exactly the same location that it occupied during scanning.

Therefore, there is a need for a surgical navigation system specifically designed for use in an arthroscopic procedure.

### SUMMARY OF THE INVENTION

Surgical navigation offers a better way to diagnose, plan and treat. FAI (femoral acetabular impingement) during hip arthroscopy. Surgical navigation for hip arthroscopy may include the following:

### Pre-Operative Planning:

A CT (Computerized Axial Tomography, or CAT), MRI (Magnetic Resonance Imaging), fluoroscopic, X-ray, ultrasound and/or other appropriate type of scan is taken of the patient's anatomy.

Registration markers may be placed in the femur and/or acetabulum before any scans are taken. These registration markers may be placed percutaneously so that they reside on or in the bone of the patient, below the surface of the skin, where they may remain in place during the patient scan, and until and during, the patient's arthroscopic procedure. The registration markers may be placed at their desired location using a needle or gun type delivery device or any other delivery device that is appropriate. Preferably, the placement of the registration marker does not leave a significant opening in the patient's skin, so that the opening, if any, may be covered with a bandage, or require only a small amount of stitching to close. The opening may be small enough that it requires no coverage at all. This approach will allow the patient to resume their normal activity after the scan without worrying about a registration marker protruding through their skin or a large wound which may become infected.

The registration markers may contain means for detection, such as radiopaque materials, electromagnetic (EM) technology, radio frequency (RF) technology, transponder technology, or other technologies so that the registration markers can be detected below the surface of the patient's skin. The registration marker may be secured to bone by a thread, barb, hook, tooth, or other fixation means. A pilot hole may be drilled in the bone prior to implanting the registration marker. The registration markers may be removable or bioabsorbable. Possible bioabsorbale materials include, but are not limited to, bioabsorbable, radiopaque polylactide (PLA 96-barium sulfate, BaSO₄), poly-L,D-lactic acid (SR-PLA 96/4) blended with barium sulfate, and the materials disclosed in U.S. Pat. Nos. 6,174,330 and 7,553,325, both of which are hereby incorporated herein by reference. The registration markers are configured so that they will be visible in a scan of the patient anatomy, and hence incorporated in the 3-D virtual model generated from that scan, so that the registration markers can be used to place the 3-D virtual model in proper registration with the real-world anatomy, and hence images from the 3-D virtual model can be placed in proper registration with images of the real-world anatomy.

The scan data is aggregated into a 3-D data set, and then software is used to generate a 3-D virtual model of the patient's anatomy, e.g., the acetabulum and femur in the case of hip surgery. Preferably the software is configured to identify different tissue structures and create a virtual object representative of each different tissue structure, e.g., a first virtual object to represent the femur, a second virtual object to represent the acetabulum, a third virtual object to represent the labrum, etc. Each of these virtual objects occupies a space within the virtual model which corresponds to the space that their real-world counterparts occupy in the patient anatomy. The virtual objects may be represented as polygonal surface models (i.e., wire mesh models) or other types of models known to those skilled in the art (e.g., volume models).

In one aspect of the invention, the surgeon can utilize the 3-D virtual model to diagnose CAM/pincer FAI with dynamic biomechanical simulation of the virtual femur relative to the virtual acetabulum. In this approach, the virtual femur is manipulated (e.g., flexed, rotated, abducted/adducted, or other motion) in the 3-D virtual model to confirm a FAI diagnosis and perform pre-operative planning of CAM/pincer decompression. Location, area and depth of decompression are identified (e.g., with respect to the 3-D virtual model) and saved electronically to be retrieved during the arthroscopic procedure. This virtual manipulation mimics the anatomy's real biomechanics by basing rotation of the femur on a pre-determined center of rotation within the acetabular cup. In one embodiment, the virtual femur is flexed and internally rotated relative to the virtual acetabulum. This is a typical diagnostic technique used to diagnose FAI in real-world diagnosis, and it is equally applicable in a virtual diagnosis effected using the 3-D virtual model. At the point that the virtual bones touch one another in the 3-D virtual model (i.e., the point of impingement), the degree of flexion and internal rotation may be recorded. The virtual femur may be further flexed and internally rotated within the 3-D virtual model through the desired range of motion. Based on the interference detected between the virtual femur and virtual acetabulum, the 3-D virtual model may be used to determine the area, volume and/or location of the bone or other tissue to be removed to resolve patient FAI. Thus, the location, area and depth of decompression are identified with respect to the 3-D virtual model and may be saved electronically to be retrieved later. The 3-D virtual model may also enable the surgeon to alter the identified treatment characteristics to best match the type of surgical instrument that will be used for decompression, or any other mitigating factors in the anatomy, patient make-up, and/or surgical plan.

### Intra-Operative Procedure:

The 3-D virtual model is placed into registration with the real-world anatomy, preferably by using the aforementioned arthroscopic registration markers, although this may also be done using an EM (electromagnetic) probe or the like. In other words, the coordinate system of the 3-D virtual model is placed into registration with the coordinate system of the real world anatomy—as a result, a location in one system may be properly correlated with the corresponding location in the other system. Since a leg is often manipulated during an arthroscopic procedure, placing registration markers in bone or hard tissue is preferable, since this will ensure dynamic tracking of the real bone (e.g., acetabulum, femur, etc.) in real-time. Conversely, using an EM (electromagnetic) probe or the like requires re-registration of the 3-D virtual model to the real-world anatomy every time the real-world anatomy moves. Furthermore, using registration markers is generally preferable over using an EM (electromagnetic) probe or the like, since the registration markers are in position during the scan and hence automatically incorporated, with great precision, directly into the 3-D virtual model, thereby permitting substantially perfect registration between the 3-D virtual model and the real-world anatomy, whereas the EM (electromagnetic) probe or the like involves touching the probe to known anatomical landmarks, a process which has inherently less precision than fixed-position registration markers.

Surgical instruments (e.g., burrs, cannulas, spacers, etc.) may have integrated registration markers (including detection means such as radiopaque materials, electromagnetic (EM) technology, radio frequency (RF) technology, transponder technology, or other technologies) so that the surgical instruments may be tracked during the arthroscopic procedure, whereby actual decompression may be tracked relative to the planned decompression. In other words, the real-world surgical instruments are tracked so that their positions may be considered in the context of the 3-D virtual model. Since the 3-D virtual model may have identified the bone regions which are to be removed during decompression, this tracking of the real-world surgical instruments allows a determination of the bone removal effected by those instruments with respect to the decompression regions previously identified on the 3-D virtual model. By way of example, the area of decompression may be identified on the 3-D virtual model during pre-operative planning. During the actual procedure, the burr is tracked so that its location is known vis-à-vis the real world anatomy and the 3-D virtual model. Hence, the bone removed by the burr can be limited to the targeted area of decompression.

In addition to the foregoing, real-world surgical implants (e.g., anchors) may also be tracked so that their real-world disposition can be compared with their planned disposition on the 3-D virtual, model.

Additionally, the arthroscope is preferably also tracked with a registration marker, so that the real-world orientation of the arthroscope can be identified. This allows the image rendered from the 3-D virtual model to be placed into proper registration with the real-world image obtained by the arthroscope. More particularly, by knowing the optics of the real-world arthroscope, the image rendering engine of the 3-D virtual model can generate an image of the 3-D virtual model which has similar optical characteristics to the real-world arthroscope (e.g., field of view, focal length, etc.). Furthermore, by tracking the real-world position of the arthroscope, the “camera position” of the image rendering engine in the 3-D virtual model can be specified for a corresponding position in the 3-D virtual model, so that the image generated by the 3-D virtual model will correspond (optically and positionally) to the real-world image generated by the arthroscope.

Using a combination of tracked real-world anatomy, a 3-D virtual model, placed in registration with the tracked real-world anatomy, a tracked arthroscope, and tracked instruments, the 3-D virtual model can be used to guide decompression with bone-cutting tools (e.g., burrs). In one preferred form of the invention, the surgeon may see a composite image on the arthroscopic monitor, e.g., a virtual world image (provided by the 3-D virtual model) superimposed on the real-world image (provided by the arthroscope or other technology). By way of example but not limitation, the virtual world image may be a semi-transparent overlay on top of the real-world image, with the virtual world image comprising an indicator of the specific bone which is to be removed during decompression. The surgeon may or may not directly control instrument movement. Thus, in one form of the invention, the surgeon directly controls the instrument and observes, visually, how the location of the real-world instrument correlates against the model's indication of where the bone is to be decompressed. In another form of the invention, the instrument may be controlled by a robot which has been pre-programmed to remove the bone which is to be decompressed. In this situation, the coordinate system of the robot must be placed into proper registration with the coordinate system of the real-world anatomy, in order for the robot to accurately remove the desired bone.

Where registration markers are used to identify the location of the real-world anatomy, those registration markers need to be secured to the anatomy. Typical anatomical structures include rigid tissues such as bone. As noted above, once the registration marker has been secured to the tissue and scanned with that tissue so that it is automatically incorporated into the 3-D virtual model, the registration marker may be used to place the 3-D virtual model into proper registration with the real-world anatomy, whereupon the 3-D virtual model can used to guide the real-world instruments in order to treat patient FAI.

The registration markers may be placed before or after scanning the anatomy. In general, it is preferred that the registration markers be placed before scanning the anatomy so that: they are automatically incorporated into the 3-D virtual model. If the registration markers are placed into the anatomy after scanning, they may be placed as follows: an arthroscopic instrument may place the registration marker onto the anatomical structure. These anatomical structures may include the femur and the acetabulum. One or more registration markers may be placed on each structure in order to provide a continuous reference to the anatomy. Preferably more than one registration marker is placed into the anatomy so as to facilitate fast and accurate registration of the 3-D virtual model and the real-world anatomy. An external registration system determines the position and/or orientation of the anatomical structure via the registration markers. The registration markers may use detection technology such as radiopaque materials, electromagnetic (EM) technology, radio frequency (RF) technology, transponder technology, or other technologies. The registration marker may be secured to the bone by a thread, barb, hook, tooth, or other fixation. A pilot hole may be drilled prior to implanting the marker.

The registration marker may be removable from the anatomical structure (e.g., bone), typically after the arthroscopic procedure is completed. Removal may include unscrewing or disengaging the registration marker from the bone. The removal may be done arthroscopically.

Where a probe is used to determine the position and orientation of the anatomical structure, the probe may use detection technology such as radiopaque materials, electromagnetic (EM) technology, radio frequency (RF) technology, transponder technology, or other technologies in order to track the location of the probe. The probe is touched against one or more known anatomical landmarks to identify the real-world position of those anatomical landmarks and, as a result, the real-world position of the anatomical structure carrying those anatomical landmarks. These anatomical landmarks can then be located on the 3-D virtual model (since they were present during scanning) and, as a result, by placing the anatomical landmarks of the 3-D virtual model into correspondence with the anatomical landmarks of the real-world anatomy, the 3-D virtual model can be placed into registration with the real-world anatomy. Of course, any movement of the real-world anatomy after such probe detection requires that the probe detection be repeated so as to determine the new location of the anatomy. Alternatively, registration markers may be inserted into the anatomy after the probe has been used to identify the location of the anatomy, so that registration can be maintained even if the anatomy is moved Or the registration markers may be inserted into the anatomy after scanning but prior to using the probe to identify the location of the anatomy. Possible landmark points for the femur include, but are not limited to, articular locations (e.g., femoral head bone surface areas), peripheral compartment locations (e.g., femoral, neck), and extra-articular locations (e.g., greater trochanter, trochanteric crest, etc.), etc. Possible landmark points for the pelvis include, but are not limited to, points on the anterior superior iliac spine, the acetabulum rim, etc.

Additionally, the virtual anatomic model (i.e., the 3-0 virtual model) that was created pre-operatively is placed into registration with the real-world anatomy. Once the registration markers are anchored in the anatomic structures, or known anatomical landmarks have been identified, the 3-D virtual model is placed into registeration with the real-world anatomy. This may be done by correlating the position of the registration markers in the 3-D virtual model with the location of the registration markers in the real-world anatomy. This may also be done by identifying the location of a specific anatomic feature (such as a pronounced anatomical landmark) present in both the 3-D virtual model and the actual anatomy. Alternatively, a probe may be placed on one or more locations to reference the anatomy to the virtual anatomic model. By identifying corresponding locations between the virtual model and the real-world anatomy, the two systems are placed into registration with one another. Surgical tools are preferably also tracked. In this way, the movement of surgical tools may be tracked and modeled on the 3-D virtual model. This allows the treatment (e.g., the amount of bone removed) to be tracked and compared to the pre-operative plan which is visible on the 3-D virtual model. This allows the surgeon to determine if a sufficient amount of bone has been removed to treat the patient's FAI.

A tool, such as a burr, is placed into the joint through an access cannula. The cannula, and any tools used, may include one or more registration markers. The registration marker on the cannula provides the location of the cannula in the real world and, as a result, where the 3-D virtual model is in registration with the real-world anatomy, allows a virtual cannula to be identified in the 3-D virtual model. It may also enable a robotic arm to guide a surgical instrument to and through the specific cannula. It may also prevent the robotic arm from damaging tissue by providing information of the proper location of the cannula in the real-world anatomy, which may be correlated with the coordinate system of the robotic arm. Additionally, the registration marker may provide a unique identification of the cannula; an example of this would be its size, such as diameter.

The surgical navigation system may also provide a means to place an anchor, e.g., to re-fixate the labrum to the acetabulum. The drill, punch, guide, anchor delivery tool and/or the anchor itself may all have registration markers that are tracked by the navigation system. This may, for example, enable preferable and/or safer placement of the anchor. For example, 3-D tracking of the anchor placement may prevent: the anchor from piercing through the articular surface of the acetabulum. Additionally, it may be used to obtain more accurate positioning of the anchor relative to the labrum and acetabulum.

A balloon or other spacer that creates or maintains the joint distraction during a procedure may also include a registration marker to identify its location and/or orientation with surgical navigation during hip arthroscopy. This may provide information about: the direction and/or degree of joint distraction. It may also be used to ensure that the real-world balloon is placed in the most appropriate location for the desired joint distraction (which may or may not be identified via the 3-D virtual model). Identification of the balloon's location in the 3-D virtual model may also prevent a robotically manipulated tool from contacting the balloon and causing damage to the balloon.

Other tools used in an arthroscopic procedure may include, but are not limited to, burrs, cannulas, anchors, scopes, graspers, shavers, microfracture tools, blades, scissors, guidewires, retractors, switching sticks, balloon spacers or other spacers etc. Tracking means may also be provided on the tool so that the real-time position of the tool is always known with respect to the virtual model.

The surgical navigation system may include a fixture to mount the patient's leg. The fixture allows manipulation of the leg and may include registration markers. Additional arthroscopic access to pathology may be enabled by moving the femur. Tracking this movement via the leg fixture allows the surgical navigation system to guide tools to treat pathology (for example, remove bone to relieve CAM impingement) more effectively.

The surgical navigation system may include a fixture, or cuff, to be placed around the leg and another fixture, or cuff, to be placed on the pelvis. These fixtures or cuffs may include registration markers that can be used to track the position of the femur relative to the pelvis. These fixtures or cuffs may have features which can locate to bone proturbances such as the iliac spine and greater trochanter.

The above surgical navigation system may also be utilized for a mini-open, or partial arthroscopic, procedure. In this embodiment, one or more cannulas are placed into the joint. Additionally, a small incision is made to access the joint space. An arthroscope may be placed through the cannula. Additionally, a balloon spacer, other spacer, retractor or other tool may be placed through a second cannula. A cutting tool, such as a burr, may be placed through the small incision to treat pathology. One or more of these instruments and cannulas may include registration markers. Additionally, one or more registration markers may be placed onto anatomic structures directly through the small incision. A robotic arm may control any tools.

Any embodiments of the invention may also be used for other minimally invasive orthopedic procedures including, but not limited to, knee and shoulder arthroscopy.

The registration markers that were secured to the anatomy in order to place the 3-D virtual model in registration with the real-world anatomy may removed under arthroscopic guidance. Specific tools may be used to unscrew, unhook, untie or otherwise remove the registration markers from the anatomy. The registration markers may be removed in this manner whether they were placed percutaneously or arthroscopically. If desired, the arthroscopic registration markers may also be left permanently in the body.

In one preferred form of the invention, there is provided a method for performing a surgically navigated arthroscopic procedure, the method comprising:

percutaneously placing at least one registration marker on an anatomical structure at an arthroscopic site;

scanning the anatomical structure and the at least one registration marker on the anatomical structure;

creating a 3-D virtual model of the scanned anatomical structure and the at least: one registration marker on the anatomical, structure;

placing the 3-D virtual model in registration with the anatomical structure; and

performing an arthroscopic procedure using the 3-D virtual model.

In another preferred form of the invention, there is provided a method of performing a surgically navigated arthroscopic procedure, the method comprising:

scanning an anatomical structure;

creating a 3-D virtual model of the scanned anatomical structure;

analyzing the 3-D virtual model to identify tissue to be removed;

modifying the 3-D virtual model to visually identify tissue to be removed, whereby to create a modified 3-D virtual model;

placing the modified 3-D virtual model in registration with the anatomical structure; and

performing an arthroscopic procedure using the modified 3-D virtual model.

In another preferred form of the invention, there is provided a balloon spacer comprising tracking means.

In another preferred form of the invention, there is provided an anchor comprising tracking means.

In another preferred form of the invention, there is provided a method for positioning an anchor in an anatomical structure, comprising:

scanning an anatomical structure;

creating a 3-D virtual model of the scanned anatomical structure;

placing the 3-D virtual model in registration with the anatomical structure; and

placing the anchor in the anatomical structure using the 3-D virtual model.

In another preferred form of the invention, there is provided a method for accessing the central compartment of a hip, comprising:

scanning an anatomical structure;

creating a 3-D virtual model of the scanned anatomical structure;

placing the 3-D virtual model in registration with the anatomical structure; and

guiding a tracked needle into the central compartment of a hip using the 3-D virtual model.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts and further wherein:

FIGS. 1A-1B show the anatomy of the human hip and joint, area;

FIGS. 2A-2C show a registration marker being placed percutaneously;

FIG. 3 shows various locations where registration markers may be placed;

FIG. 4 shows various locations where registration markers may be placed;

FIGS. 5A-5B show 3-D virtual images of the femoral head of a hip joint with registration markers in place;

FIGS. 6A-6B show 3-D virtual images including designation of bone material to be removed to reduce impingement;

FIG. 7 shows a cannula;

FIG. 8 shows a balloon spacer;

FIG. 9 shows an anchor delivery system;

FIGS. 10 and 11 are schematic views showing CAM-type femoroacetabular impingement;

FIGS. 12 and 13 are a schematic views showing pincer-type femoroacetabular impingement; and

FIG. 14 is a flowchart illustrating an arthroscopic procedure using surgical navigation.

### DETAILED DESCRIPTION OF THE PREFERRED EMBODIMENTS

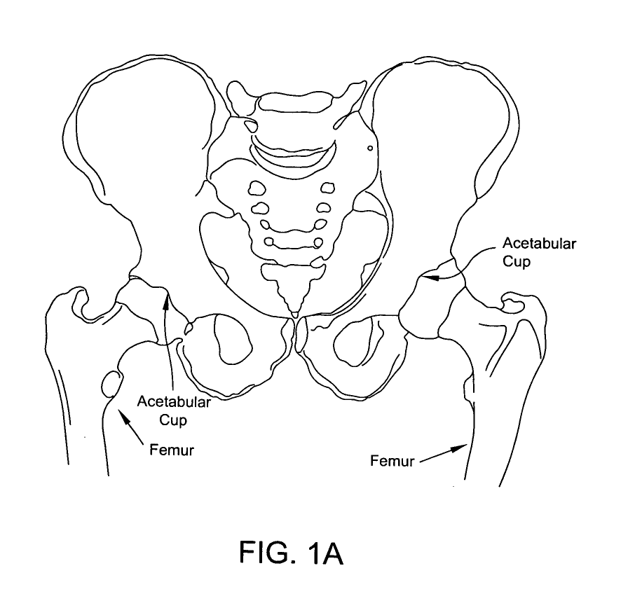

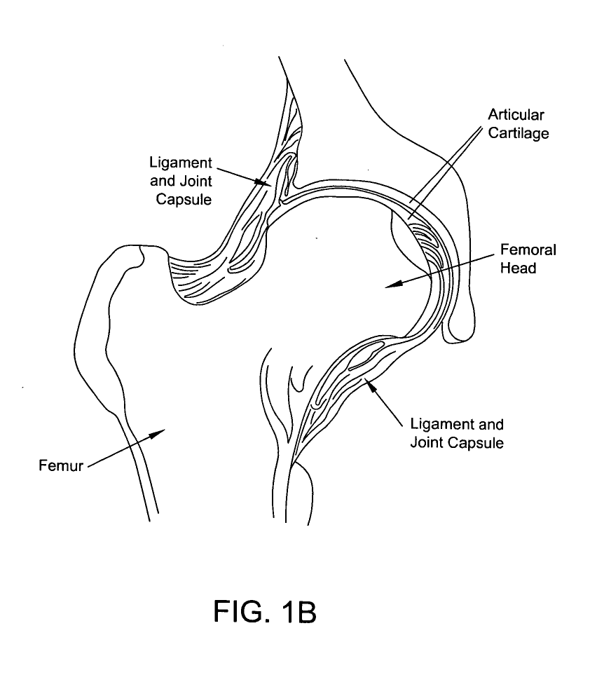

FIG. 1A is a diagram of the human hip. Each hip joint is made up of a femur seated within an acetabular cup. FIG. 1B shows a close-up of the femoral head seated within the acetabular cup. In a normal hip joint, the femoral head moves freely within the acetabular cup and has a full range of motion. Acetabular impingement exists where either the femur or the acetabular cup, or both, is abnormally shaped, decreasing the range of motion of the hip joint through impingement.

Where there is impingement, bone and/or other tissue may be removed to reduce the impingement and thereby increase range of motion. Preferably the impinging tissue is removed using arthroscopic techniques, and this is preferably performed under surgical navigation guidance. This arthoscopic approach reduces pain and recovery time for the patient and increases accuracy and outcome for the surgeon. This arthroscopic approach may also reduce surgery time.

Before the arthroscopic procedure is initiated, the hip area is scanned to produce a 3-D virtual model of the anatomy that will be used for surgical navigation during the subsequent arthroscopic procedure. Preferably arthroscopic registration markers are applied to the anatomy prior to scanning so that the 3-D virtual model includes the registration markers, which can then be used later to align, or register, the 3-D virtual model with the real-world anatomy (and hence align images generated from the 3-D virtual model with arthroscope images of the real-world anatomy). These registration markers are preferably arthroscopic registration markers placed on the bones or other tissue of the patient prior to the scanning. Alternatively, registration can be achieved by using known anatomical landmarks visible in the 3-D virtual model and reachable in the real-world anatomy with a tracked probe, however, such an arrangement is generally not preferred since any real-world movement of the anatomy requires that the real-world location of the anatomical landmarks be re-acquired using the tracked probe.

If registration markers are to be placed on the bones of the patient prior to scanning, preferably they are placed percutaneously, meaning below the skin, so that the patient may go about his or her regular activities during the period of time after the scan is conducted and before the arthroscopic procedure is performed. Preferably, the placing of the registration markers leaves little or no wound on the patient so the risk of infection is low.

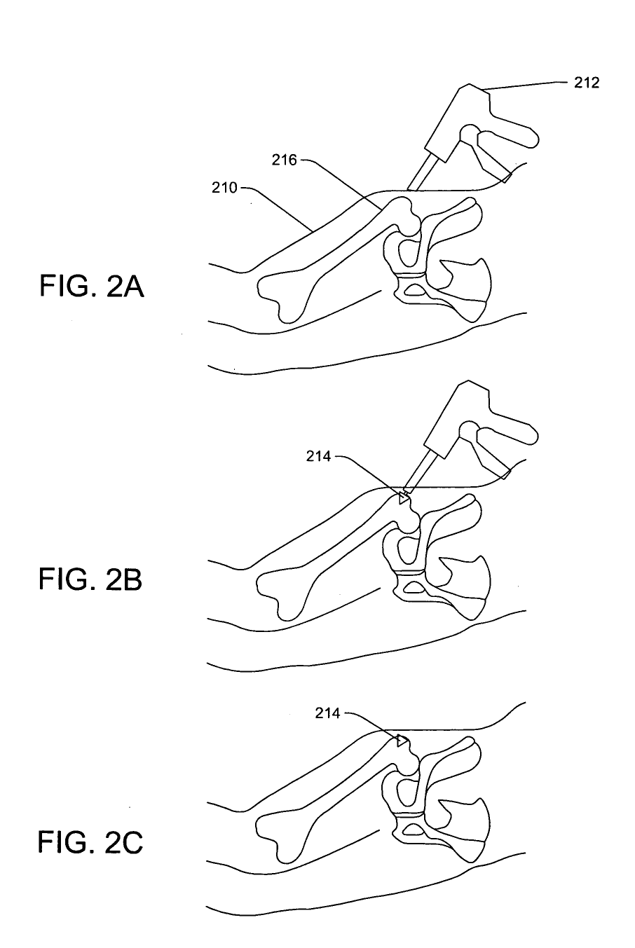

FIGS. 2A-2C show one method of marker placement. In this method, the registration markers are loaded into a marker delivery device 212. The marker delivery device may be a needle or gun type device or any other suitable device for delivering the registration markers percutaneously, below skin 210. FIG. 2A shows marker delivery device 212 up against the skin of the patient. The marker delivery device is aimed at bone 216. In FIG. 2B, the marker delivery device is shown delivering marker 214 onto or into the bone of the patient. This delivery is performed percutaneously, or through the skin. FIG. 2C shows the registration marker in place on or in the bone after the delivery device has been removed. The registration marker preferably does not protrude through the skin of the patient and leaves only a small wound in the skin of the patient.

The registration markers may be placed just prior to the scanning procedure, or they may be placed in a separate procedure some time before the scanning of the patient. In either case, because the registration markers are in place prior to scanning, they will be automatically incorporated into the scan and hence automatically incorporated into the 3-D virtual model created from the scans. The registration marker is made out of a biocompatible material and may be left in place for some time without causing infection or discomfort. The registration markers may be made of a biodegradable material, but are preferably made of a more stable material which may be removed later, i.e., during the arthroscopic procedure. The registration marker is preferably made out of metal and/or polymer and/or other suitable biocompatible material and may contain technology such as radiopaque (EM) materials, radio frequency (RF) materials, transponder technology, or other technologies so that they are detectable below the patient's skin. The registration marker may be secured to the bone by a thread, barb, hook, tooth, or other fixation. A pilot hole may be drilled prior to implanting the registration marker. The registration markers may be removable or bioabsorbable. Possible bioabsorbale materials include, but are not limited to, bioabsorbable radiopaque polylactide (PLA 96-barium sulfate, BaSO₄), poly-L,D-lactic acid (SR-PLA 96/4) blended with barium sulfate, and the materials disclosed in U.S. Pat. Nos. 6,174,330 and 7,553,325, both of which are incorporated herein by reference. The registration markers will be visible in a scan of the body so that their location will be automatically incorporated into the 3-D virtual model of the anatomy, whereby the 3-D virtual model can later be placed into registration with the real-world anatomy by aligning the locations of the registration markers on the 3-D virtual model with the locations of the actual registration markers in the actual anatomy.

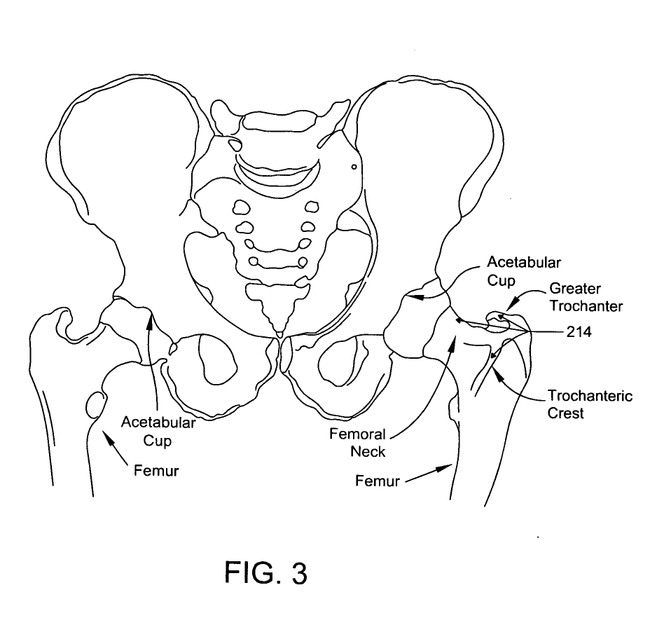

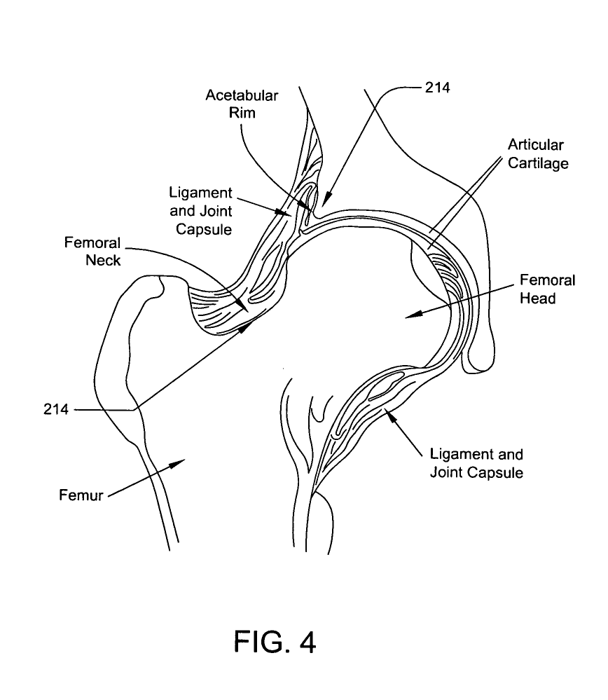

The registration markers are placed preferably on or in the bones of the hip joint. FIGS. 3 and 4 show some possible areas of the hip joint where registration markers 214 may be placed. Preferably, the registration markers are placed in locations which will later be accessible by arthroscopy so that they can be removed arthroscopically.

The hip area is scanned using CT, MRI, fluoroscopic, X-ray, ultrasound or other appropriate scanning technology. In addition to the anatomy of the patient, the location and possibly the orientation of the registration markers is identifiable in the scans of the patient.

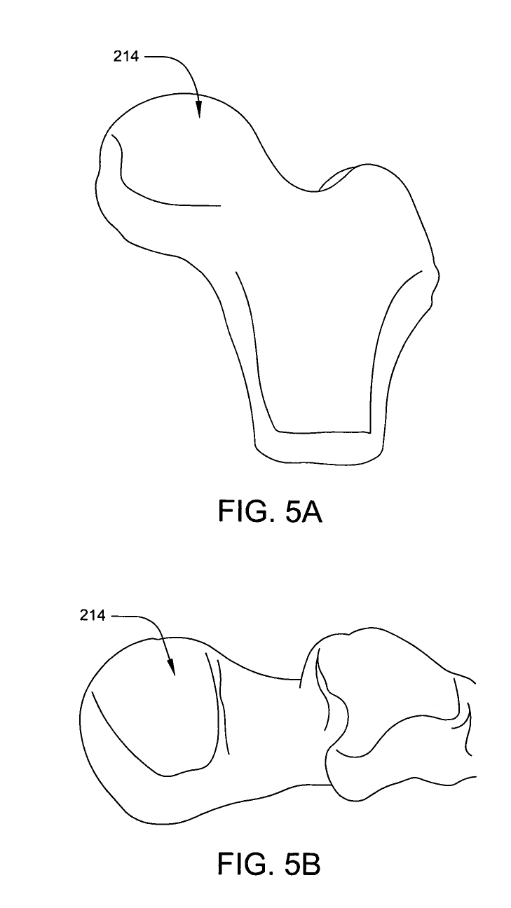

Using the scans of the patient, a 3-D virtual model is created of the hip joint. This 3-D virtual model includes the patient's anatomy as well as the location and possibly the orientation of the registration markers. FIGS. 5A and 5B show a 3-D virtual model of a femur with marker 214 visible. As is well known to those skilled in the art of computer modeling, the 3-D virtual model is able to be rotated and manipulated as is shown in FIGS. 5A and 5B.

The surgeon can use the 3-D virtual model to diagnose CAM/pincer FAI with dynamic biomechanical simulation of the virtual femur relative to the virtual acetabulum. The virtual femur can manipulated (flexed, rotated, abducted/adducted, etc.) relative to the virtual acetabulum in the 3-D virtual model to confirm a FAI diagnosis and perform pre-operative planning of CAM/pincer decompression. Location, area and depth of decompression can be identified and saved electronically. This manipulation of the virtual objects in the 3-D virtual model mimics the biomechanics of the actual anatomy by basing rotation of the virtual femur on a pre-determined center of rotation. In one embodiment, the virtual femur is flexed and internally rotated relative to the virtual acetabulum. At the point, that the virtual bones touch in the 3-D virtual model (i.e., the point of impingement), the degree of flexion and internal rotation may be recorded. The virtual femur may be further flexed and internally rotated to the desired range of motion. Based on the interference of the virtual femur and virtual acetabulum, the 3-B virtual model may detect the area, volume and location of the bone or tissue to be removed in order to treat FAI.

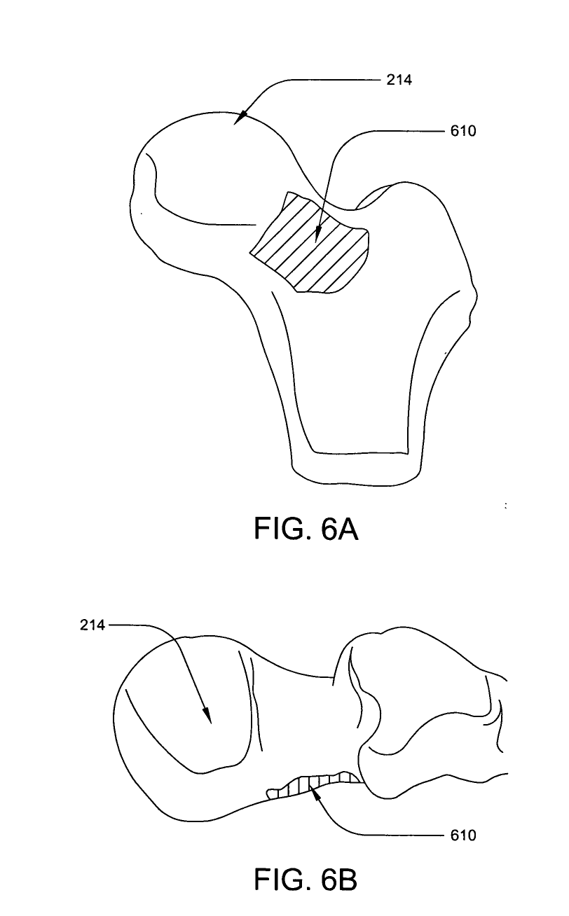

FIGS. 6A and 6B show a virtual 3-B model of a femur with an area 610 of bone that is to be removed. The area 610 includes a depiction of the location, area and depth of decompression and this information may be saved electronically to be retrieved later, e.g., during the arthroscopic procedure. The 3-D virtual model may also enable the surgeon to alter the identified treatment characteristics to best match the type of cutter that will be used, or any other mitigating factors in the anatomy, patient make-up, and/or surgical plan.

The scanning of the patient may be performed on the same day as, or some time before, the scheduled arthroscopic procedure. Allowing some time between the scanning and the arthroscopic procedure allows the physician to create and analyze the 3-D model at his/her leisure. Since the registration markers used in the scanning are below the skin, the arthroscopic procedure does not need to begin immediately after the scan is performed, as would be the case if the registration markers were protruding through the patient's skin.

During the arthroscopic procedure, the 3-D virtual model is placed into registration with the real-world anatomy. This is done by aligning the location and possibly the orientation of the virtual registration markers in the 3-D virtual model with the location and possible the orientation of the registration markers in the real-world anatomy. Alternatively, the 3-D virtual model can be placed into registration with the real world anatomy by means of known anatomic landmarks which can be located on the 3-D virtual model and accessed in the real-world anatomy with a tracked probe. Once the 3-D virtual model has been placed into registration with the real-world anatomy, a composite image can be generated which combines virtual images of virtual objects in the 3-D virtual model with real images of the real-world objects (e.g., anatomy, instruments, etc.).

Several tools may be used during the arthoscopic procedure including, but not limited to, the endoscope itself, burrs, cannulas, anchors, balloon spacers or other spacers, cuffs, fixtures etc. Tracking marker means may be provided on the tools so that the real-time position of a tool is known with respect to the 3-D virtual model.

As the procedure progresses and tissue is removed from the hip joint, preferably the physician is able to view the progress of the tissue removal via the real-world images of the arthroscope and the virtual images generated by the 3-D virtual model. Preferably the 3-D virtual model indicates to the physician what tissue needs to be removed and how to best access that tissue. The 3-D virtual image may be visible to the physician separately from the real-world arthroscope image (e.g., on separate monitors), or more preferably, the arthroscope images and the 3-D virtual model images may be overlaid each other in a composite image. The physician preferably can control how the images are shown so that he/she can best navigate and perform the procedure.

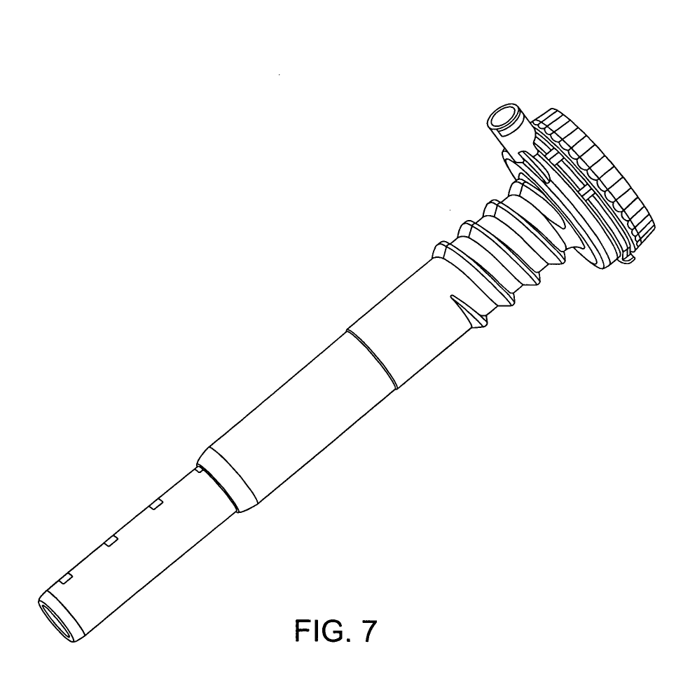

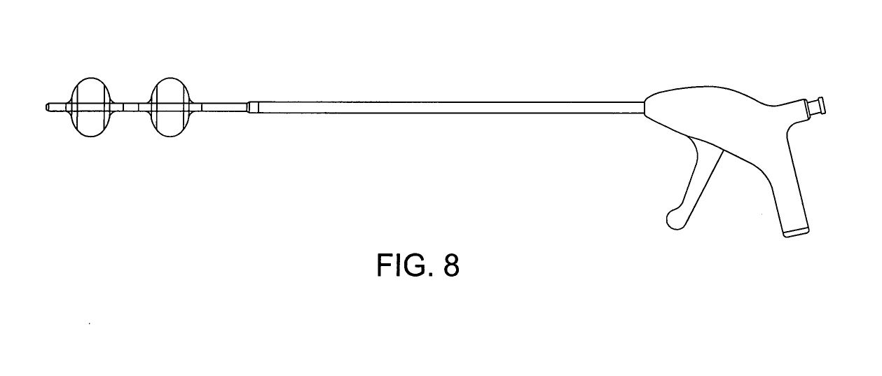

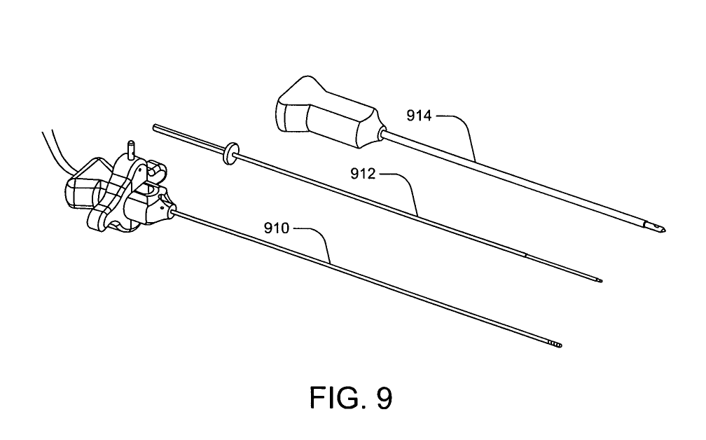

FIGS. 7-9 show some examples of tools that may be used during an arthroscopic procedure conducted under surgical navigation. FIG. 7 shows a cannula which may be used to access the hip joint site. FIG. 8 shows a balloon spacer which may be used for joint distraction and/or space maintenance, allowing access between the acetabular cup and the femoral head. FIG. 9 shows an anchor delivery system which may be used during the arthroscopic procedure. Anchor delivery system guide 914 is used to locate the place where the anchor will be delivered. After the guide is in place, drill 912 is used through the guide to drill a hole into the bone or other tissue. While the guide remains in place, the drill is removed and anchor inserter 910 is introduced into the guide. The anchor inserter then places the anchor into the hole drilled by drill 912. Registration markers may be placed on any of the components of the anchor delivery system, and/or the anchor itself.

Note that the anchor delivery system shown in FIG. 9 could also be modified to be used as a percutaneous or arthroscopic registration marker delivery device. In other words, the anchor delivery system can be used to deliver a registration marker instead of an anchor.

Other tools used in an arthroscopic procedure may include, but are not limited to, burrs, cannulas, anchors, balloon spacers or other spacers, external cuffs or fixtures, etc. Tracking means may also be provided on the tool so that the real-time position of the tool is always known with respect to the 3-D virtual model.

After the 3-D virtual model and the real-world anatomy have been placed into registration with one another, so that their respective images can be placed into registration with one another, and preferably after all movement of the anatomy has ceased, the registration markers may be removed. Their removal can be done before or after the impingement tissue is removed from the hip joint; however, since it it frequently desirable to move the real-world anatomy during the decompression, it is generally desirable to remove the registration markers only after of the decompression has been completed. Preferably, the registration markers are removed arthroscopically. Removal may be performed by unscrewing or disengaging the registration markers from bone or by other means. If the registration markers are biodegradable, they do not need to be removed. Alternatively, if desired, the registration markers can be left permanently in the body.

The above surgical navigation system may also utilized for a mini-open, or partial-arthroscopic, procedure. In this embodiment, one or more cannulas are placed into the joint. Additionally, a small incision is made to access the joint, space. An endoscope may be placed through the cannula. Additionally, a balloon spacer, other spacer, retractor or other tool may be placed through a second cannula. A cutting tool, such as a burr, may be placed through the small incision to treat pathology. One or more of these instruments and cannulas may include registration markers. Additionally, one or more registration markers may be placed onto anatomic structures directly through the small incision. A robotic arm may control the burr tool.

Any embodiments of the invention may also be used for other minimally invasive orthopedic procedures including, but not limited to, knee and shoulder arthroscopy or surgery.

### Example 1

As noted above, hip arthroscopy is becoming increasingly more common in the diagnosis and treatment of various hip pathologies. However, due to the anatomy of the hip joint and the pathologies associated with the same, hip arthroscopy is currently practical for only selected pathologies.

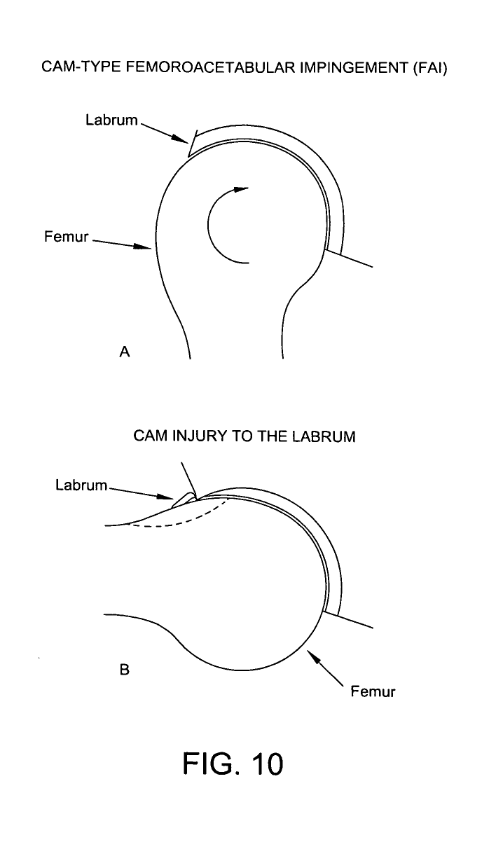

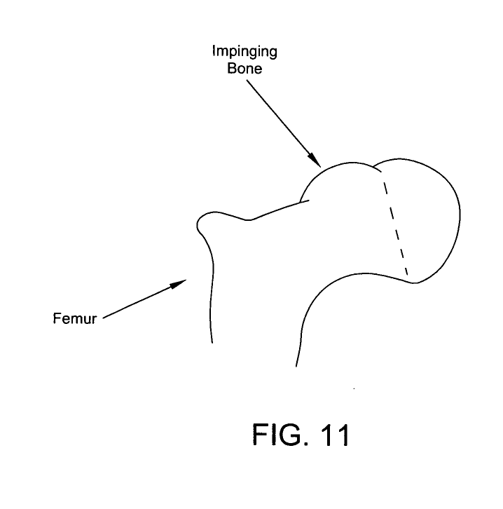

One procedure which is sometimes attempted arthroscopically relates to femoral debridement for treatment of cam-type femoroacetabular impingement (i.e., cam-type FAI) and/or acetabular debridement for treatment of acetabular femoroacetabular impingement (i.e., pincer-type FAI). More particularly, with cam-type FAI, irregularities in the geometry of the femur can lead to impingement between the femur and the rim of the acetabular cup. Treatment for cam-type FAI typically involves debriding the femoral neck and/or head, using tools such as burrs, to remove the bony deformities causing the impingement. See FIGS. 10 and 11. In this respect it should be appreciated that it is important to debride the femur carefully, since only bone which does not conform to the desired geometry should be removed, in order to assure positive results as well as to minimize the possibility of a bone fracture after treatment, where too much bone is removed.

For this reason, when debridement is performed as an open surgical procedure, surgeons frequently use pre-shaped curvature templates to guide them in removing the appropriate amount of bone.

However, when the debridement procedure is attempted arthroscopically, conventional curvature templates cannot be passed through the narrow keyhole incision, and hence debridement templates are not available to the surgeon for reshaping the bone surface. As a result, the debridement must be effected freehand. In this setting, it is generally quite difficult for the surgeon to determine exactly how much bone should be removed, and whether the shape of the remaining bone has the desired geometry.

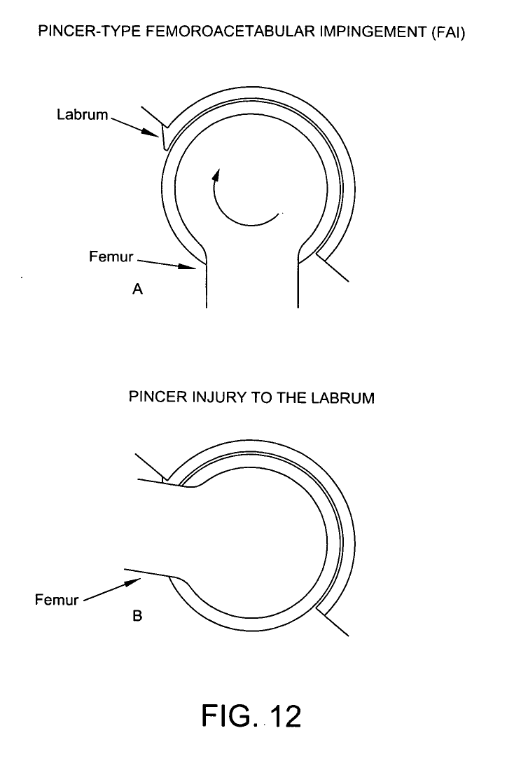

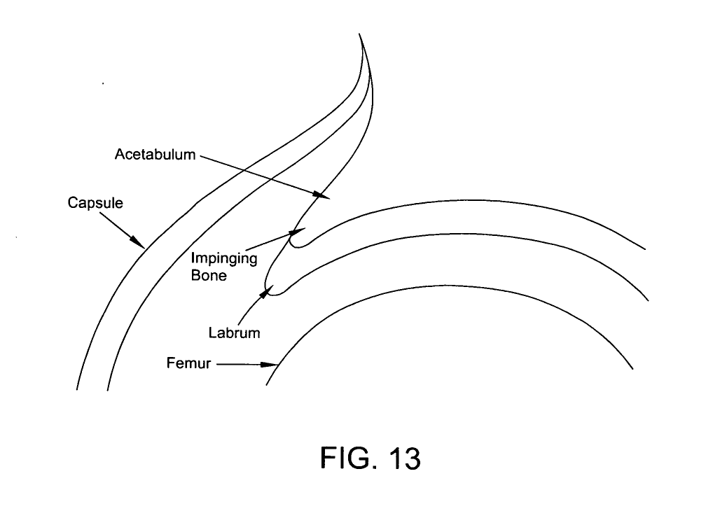

Similar problems occur with pincer-type femoroacetabular impingement (i.e., pincer-type FAI). More specifically, with pincer-type FAI, irregularities in the acetabulum can lead to impingement between the femur and the rim of the acetabular CUD. Treatment for pincer-type FAI typically involves debriding the acetabular rim, using tools such as burrs, to remove the bony deformities causing impingement. See FIGS. 12 and 13. However, when debridement is to be effected arthroscopical for pincer-type FAI, the debridement must be effected freehand. In this setting it is generally quite difficult for the surgeon to determine exactly how much bone should be removed, and whether the remaining bone has the desired geometry.

The present invention provides a solution to this problem, by permitting an arthroscopic debridement procedure to be performed using surgical navigation to safely and accurately rectify cam-type FAI and; or pincer-type FAI.

More particularly, arthroscopic surgery is typically conducted through small incisions, with the working ends of the instruments being disposed at the interior surgical site and with the handles of the instruments remaining outside the body. Visualization is obtained by using one or more endoscopes which are inserted into the body, with the surgeon watching a monitor so as to see the interior surgical site.

The present invention provides the ability to combine computer modeling of the patient's anatomy with image merging from the endoscope so as to provide the surgeon with visual cues to guide the arthroscopic procedure. Alternatively, audio cues or force-feedback (tactile) cues, etc. can be provided to the surgeon to help guide the surgery.

More particularly, in accordance with the present invention, the patient's anatomy is first scanned using an appropriate scanning technology (e.g., CT, MRI, etc.) so as to acquire slices of 2-D data (the “2-D data”).

Next, the slices of 2-D data are merged so as to create a 3-D data set reflective of the patient's actual anatomy (the “3-D data set”).

The 3-D data set is then processed so as to create a computer model of the patient's hip anatomy (the “3-D virtual model”). In other words, the voxel intensity values in the 3-D data set are segmented so as to identify specific anatomical structures, and then a computer model of these anatomical structures (i.e., the 3-D virtual model) is created. By way of example but not limitation, the 3-D virtual model may be a polygonal surface model of the patient's anatomy.

In accordance with one preferred form of the invention, registration markers are percutaneously secured to the structures of the joint prior to scanning, so that the registration markers are automatically incorporated with the 2-D data set, and hence into the 3-D data set, and hence into the 3-D virtual model.

Next, the 3-D virtual model is processed so as to create a biomechanical model of the joint (the “biomechanical model”). More particularly, the 3-D virtual model is used to create a biomechanical model of the joint which can be used to analyze the biomechanics of the joint, in both static and dynamic conditions.

The biomechanical model is then used to identify the regions of bone which need to be removed in order to eliminate impingement. In other words, the biomechanical model is used to identify where impingement will occur as the joint is moved through a normal range of motion, and where bone should be removed from the femur and/or the acetabulum in order to eliminate impingement.

Once the regions of bone which are to be removed have been identified, the 3-D virtual model is then modified so as to visually identify the regions of bone which are to be removed (the “modified 3-D virtual model”). In essence, the model is “marked” so as to show where debridement is needed in order to eliminate impingement.

Next, the modified 3-D virtual model is placed into registration with the real-world anatomy, by aligning the registration markers in the modified 3-D virtual model with the arthroscopic registration markers in the real-world anatomy.

The surgeon then initiates arthroscopic surgery, i.e., the joint is distracted, and the surgical instruments (including arthroscope) are inserted into the joint through one or more keyhole incisions.

Next, the surgeon turns on the arthroscope so as to generate the live (i.e., real-time) arthroscopic image of the interior of the joint (the “scope image”). At the same time, the modified 3-D virtual model is accessed by an image rendering engine of the sort well known in the computer modeling art so as to generate an initial image of the modified 3-D virtual model (the “modified 3-D virtual model image”). Then the live scope image is electronically merged with the modified 3-D virtual model image so as to create a composite image (e.g., with the modified 3-D virtual model image superimposed, in a semi-transparent manner, on the live scope image). The composite image is displayed on a monitor to the surgeon.

It should be appreciated that the live scope image is placed in proper registration with the modified 3-D virtual model image (e.g., using fiducial markers, anatomical landmarks, etc., and preferably using the aforementioned arthroscopic registration markers), and thereafter kept in registration with one another, as the arthroscope is moved around the surgical site (e.g., using trackers). This lets the surgeon simultaneously see the live scope image and the modified 3-D virtual model image in a superimposed manner. Since the modified 3-D virtual model image visually identifies the regions of bone which are to be removed, the system effectively provides the surgeon with a visual guide which shows, in the context of the live scope image, exactly how much bone needs to be removed during the surgical procedure.

The surgeon then removes the impinging bone using the visual guide described above. More particularly, the surgeon uses the live scope image to visualize the surgical site and, guided by the modified 3-D virtual model image superimposed on the live scope image, uses a burr or other surgical device to remove the impinging bone as imaged on the modified 3-D virtual model. Thus it will be seen that the system provides the surgeon with a visual guide which aids the surgeon during the arthroscopic debridement process.

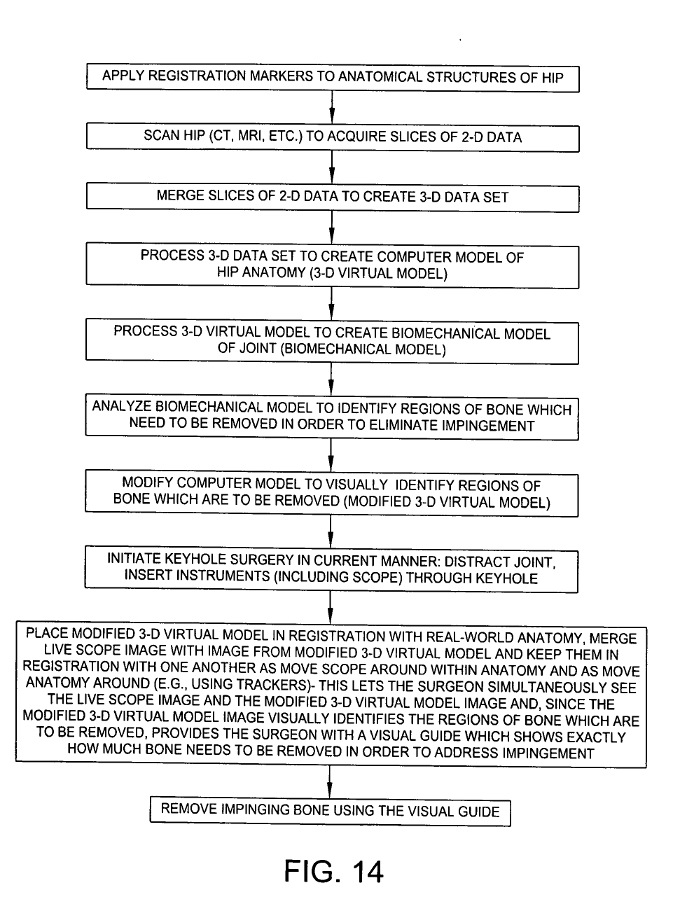

See FIG. 14, which provides a flowchart of the computer-guided arthroscopic debridement procedure discussed above.

Significantly, the surgeon always has a live camera image during surgery, thereby assuring the surgeon of the accuracy of the procedure.

Furthermore, the virtual guide is only present in electronic form on the composite image, and is never present (in a physical sense) at the surgical site.

Therefore, the surgeon always retains the option of “overriding” the virtual guide and debriding the bone according to their professional judgments.

### Example 2

In the example discussed above, the system merges the modified 3-D virtual model image with the live scope image so as to provide the surgeon a visual guide to follow during the debridement procedure. However, the modified 3-D virtual model is “blind” to the location of the debridement tool during the debridement procedure, and the virtual guide is provided solely in the form of visual markings placed on the modified 3-D virtual model prior to surgery.

Alternatively and/or additionally, it is also possible to place a “tracker” on the debridement tool, so that the system can determine the current location of the debridement tool during surgery. Furthermore, the system can integrate information about the current location of the tracked debridement tool into the modified 3-D virtual model, so as to guide the surgeon on how the debridement tool should be advanced relative to the anatomy so as to remove the impinging bone. By way of example but not limitation, in this construction, audio cues may be used to guide the surgeon during the debridement procedure, e.g., a certain audio cue can advise the surgeon to advance the tool forward, whereas another audio cue can alert the surgeon to stop tool advance. Alternatively, force-feedback elements can provide tactile cues to the surgeon, or other means can be used to provide guidance to the surgeon.

Furthermore, the system can be used to control operation of the debridement tool in accordance with its tracked position. More particularly, since the modified 3-D virtual model, is “marked” so as to indicate where debridement is required (“the target debridement zone”), and since the location of the tracked debridement tool is known vis-à-vis both the real anatomy and the modified 3-D virtual model, the system can automatically shut down the debridement tool (e.g., “turn off the power”) whenever the cutting head of the debridement tool moves outside the target debridement zone. In other words, the system can be used to activate the debridement tool only when the cutting head of the debridement tool is located within the target debridement zone. Preferably, the system is also configured to permit manual override of this feature by the surgeon.

Additionally, since different types of tissue (e.g., cortical bone, cancellous bone, cartilage, etc.) can be detected in the 2-D scan data, and hence encoded in the 3-D virtual model, and since it may be desirable to run the debridement tool in different modes (e.g., high-speed, low-speed, etc.) depending on the type of tissue being encountered, the system can also be configured to automatically adjust operation (e.g., operating speed) of the debridement tool according to the type of tissue indicated in the 3-D virtual model (and hence encountered during the actual debridement procedure).

Improved Needle Access To The Hip Joint

In arthroscopic hip surgery, it is common to initiate access to the central compartment of the hip by passing a needle through the skin and into the central compartment of the hip. This needle can then be used to facilitate introduction of a guidewire which is used to pass instruments into the central compartment. However, if not placed properly, the needle can damage tissue, e.g., scuff cartilage or pierce the labrum.

The present invention provides a solution to this problem. More particularly, by including a tracking means on the needle, the system can guide placement of the needle, i.e., once the 3-D virtual model is placed in registration with the real anatomy, the 3-D virtual model can be used to safely guide the needle into the central compartment.

### Modifications

It is to be understood that the present invention is by no means limited to the particular constructions herein disclosed and/or shown in the drawings, but also comprises any modifications or equivalents within the scope of the invention.

What is claimed is:

1. A method for performing a surgically navigated arthroscopic procedure, the method comprising:
percutaneously placing at least one registration marker on an anatomical structure at an arthroscopic site;
scanning the anatomical structure and the at least one registration marker on the anatomical structure;
creating a 3-D virtual model of the scanned anatomical structure and the at least one registration marker on the anatomical structure;
placing the 3-D virtual model in registration with the anatomical structure; and
performing an arthroscopic procedure using the 3-D virtual model.

2. A method according to claim 1 including the further step of removing the at least one registration marker.

3. A method according to claim 2 wherein the at least one registration marker is removed arthroscopically.

4. A method according to of claim 1 wherein the anatomical structure comprises a bone.

5. A method according to claim 4 wherein the bone is located in a hip joint.

6. A method according to claim 1 wherein the arthroscopic procedure comprises a decompression procedure to treat acetabular impingement.

7. A method, according to claim 1 wherein the anatomical structure is disposed at a joint.

8. A method according to claim 7 further comprising the additional step of distracting the joint with a balloon spacer which comprises a registration marker.

9. A method according to claim 1 wherein the 3-D virtual model identifies an impingement.

10. A method according to claim 9 wherein the 3-D virtual model identifies a volume of tissue to be removed in order to treat the impingement.

11. A method according to claim 10 wherein the arthroscopic procedure is performed while viewing a composite image generated by combining an arthroscope image with an image generated by the 3-D virtual model.

12. A method according to claim 10 wherein the volume of tissue is removed using a robot.

13. A method according to claim 9 wherein the impingement is identified on the 3-D virtual model by first creating a biomechanical model of the joint from the 3-D virtual model, and analyzing the biomechanical model of the joint so as to identify impingement within the joint.

14. A method according to claim 13 wherein analyzing the biomechanical model of the joint so as to identify impingement within the joint comprises manipulating virtual objects contained within the 3-D virtual model so as to identify points of impingement.

15. A method of performing a surgically navigated arthroscopic procedure, the method comprising:
scanning an anatomical structure;
creating a 3-D virtual model of the scanned anatomical structure;
analyzing the 3-D virtual model to identify tissue to be removed;
modifying the 3-D virtual model to visually identify tissue to be removed, whereby to create a modified 3-D virtual model;
placing the modified 3-D virtual model in registration with the anatomical structure; and
performing an arthroscopic procedure using the modified 3-D virtual model.

16. A method according to claim 15 wherein analyzing the 3-D virtual model to identify tissue to be removed comprises creating a biomechanical model of the anatomical structure, and analyzing the biomechanical model of the anatomical structure so as to identify tissue to be removed.

17. A method according to claim 15 wherein a registration marker is percutaneously placed on the anatomical structure prior to scanning.

18. A method according to claim 17 wherein the registration marker is removed arthroscopically at the conclusion of the arthroscopic procedure.

19. A method according to claim 15 wherein the anatomical structure is located in a hip joint.

20. A method according to claim 15 wherein the arthroscopic procedure comprises a decompression procedure to treat acetabular impingement.

21. A method according to claim 15 wherein the method includes the step of distracting the anatomical structure using a balloon spacer which incorporates a tracking marker.

22. A method according to claim 15 wherein the method includes the step of accessing the anatomical structure using a cannula which incorporates tracking means.

23. A balloon spacer comprising tracking means.

24. An anchor comprising tracking means.

25. A method for positioning an anchor in an anatomical structure, comprising;
scanning an anatomical structure;
creating a 3-D virtual model of the scanned anatomical structure;
placing the 3-D virtual model in registration with the anatomical structure; and
placing the anchor in the anatomical structure using the 3-D virtual model.

26. A method for accessing the central compartment of a hip, comprising:
scanning an anatomical structure;
creating a 3-D virtual model of the scanned anatomical structure;
placing the 3-D virtual model in registration with the anatomical structure; and
guiding a tracked needle into the central compartment of a hip using the 3-D virtual model.

27. A method according to claim 11 wherein the arthroscope image is generated by an arthroscope comprising tracking means.

28. A method according to claim 10 further comprising the use of a tracked burr to remove the volume of tissue.

29. A method according to claim 15 wherein a registration marker is arthroscopically placed on the anatomical structure after scanning.

30. A method according to claim 20 wherein decompression is effected using a tracked burr.

31. A method according to claim 30 wherein decompression is guided by at least one of a visual cue, an audio cue and a tactile cue.
